(19) United States
(12) Patent Application Publication
(10) Pub. No.: US 2009/0076258 A1
(43) Pub. Date: Mar. 19, 2009

(54) Method for synthesizing oligosaccharides and glycosylation

(76) Inventors: Klaus Buchholz, Braunschweig (DE); Jurgen Seibel, Meinhard (DE); Hans-Joachim Joerdening, Erkerode (DE); Raphael Beine, Braunschweig (DE)

Correspondence Address: GREER, BURNS & CRAIN, 300 S WACKER DR, 25TH FLOOR, CHICAGO, IL, 60606, US

(21) Appl. No.: 11/886,938

(22) PCT Filed: Mar. 22, 2006
(86) PCT No.: PCT/EP2006/060932

§ 371 Date: Dec. 14, 2007

(30) Foreign Application Priority Data
Mar. 22, 2005 (EP) 05102284.6

(51) Int. Cl. C07H 3/06; C12P 19/18

(52) U.S. Cl. 536/123.1; 435/97

(57) ABSTRACT

The invention relates to an enzymatic method for synthesizing oligosaccharides, whereby one saccharide group of a sucrose analogue each is transferred onto an acceptor molecule, for example for glycosylating a hydroxyl compound, a saccharide, peptide, or a drug. According to the inventive method, an enzymatic synthesis of β-D-fructofuranosyl-a-D-aldopyranoside is carried out in a first step, and in a second step one of the saccharide groups is enzymatically transferred onto the acceptor molecule.

The present invention relates to an enzymatic method for synthesizing oligosaccharides. The method according to the present invention is suitable for transferring a saccharide group onto a respective acceptor molecule, e.g. a hydroxyl compound, such as a saccharide, a peptide, or a drug.

In a first step, the method according to the present invention preferably uses enzymatic synthesis to produce a β-D-fructofuranosyl-α-D-aldopyranoside from saccharose, which in a second step is used as a substrate for the enzymatic transfer of one of the saccharide groups onto an acceptor molecule.

In addition to this, the present invention provides a combination of educts and enzymes with which the method according to the present invention for synthesizing oligosaccharides can be carried out.

### PRIOR ART

EP 0 130 054 discloses the use of a fructosyltransferase for transferring the fructosyl group of saccharose. For example, galactose or a glucose derivative acts as an acceptor molecule for the fructosyl group transferred from saccharose, so that a fructose derivatized at the pyranoside group is obtained. Through subsequent halogenation, a halogenated disaccharide is produced that is used as a sweetener. The transfer of the fructosyl group of fructose, using fructosyltransferase, to other acceptor molecules, such as arabinose, lactose, maltose, or glycerin, is also said to be possible.

As alternatives to oligosaccharides of human milk having a prebiotic effect, inter alia galacto-, fructo-, xylo-, and galacturonooligosaccharides are tested. Galactooligosaccharides are obtained through transglycosylation using β-galactosidase of lactose (Boehm et al., Nutrafoods 51-57 (2005)). Fructooligosaccharides are presently mainly extracted from chicory (Boehm et al., Acta Paediatrica 18-21 (Suppl. 449, 2005).

In addition, it is known to produce oligosaccharides in a manner corresponding to natural biosynthesis, by using transglycosylation of a saccharide acting as acceptor molecule, having activated saccharide derivates. These activated saccharide derivates are activated through binding of a nucleoside diphosphate to C1. This synthesis method is disadvantageous because the activated glycosides are very expensive and are not available in the large quantities required for conversions on an industrial scale.

### GENERAL DESCRIPTION OF THE INVENTION

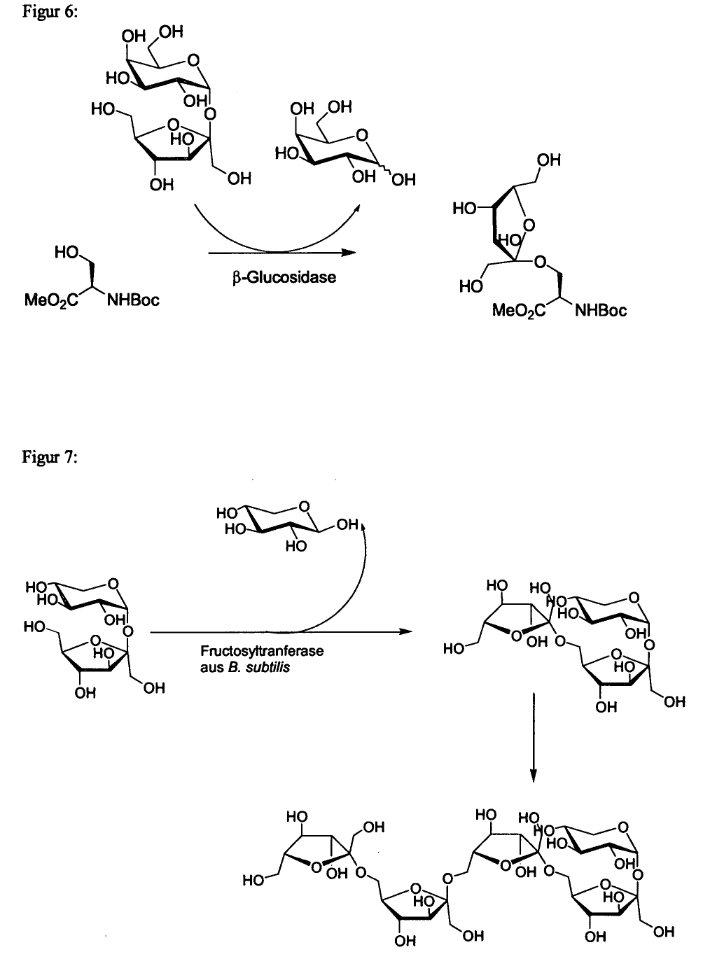

The method according to the present invention uses the binding energy of β-D-fructofuranosyl-α-aldopyranosides, generally β-D-ketofuranosyl-α-D-aldopyranosides, as substrates for the enzymatically catalyzed transfer of one of the two glycosyl groups, namely of the ketofuranyl group or of the aldopyranosyl group, onto an acceptor molecule (see for example FIGS. 4 and 7). In the preferred specific embodiment, the β-D-ketofuranosyl-α-D-aldopyranoside from which a glycoside group is transferred onto an acceptor molecule is produced by enzymatically catalyzed transfer of the glycoside group that is to be transferred onto fructose, in which process the saccharose analogue is produced and glucose is released (see for example FIG. 3). In this way, it is possible to obtain the binding energy contained in the saccharose analogue in a measure that is sufficient for the transfer of a glycoside group onto an acceptor molecule. According to the present invention, this transferred glycoside group is not originally contained in saccharose.

As an alternative to the synthesis of the analogues from saccharose, in which the natural fructosyl group is replaced by another ketofuranosyl group, or the glucoside group is replaced by another aldopyranoside group, the synthesis can also be carried out with, instead of the saccharose, another sugar that has a sufficient binding energy of a ketofuranosyl group and of an aldopyranoside in order to produce the analogue, and subsequently to enzymatically transfer one of these groups onto an acceptor. An example of such a sugar is raffinose (α-D-galactopyranosyl-(1,6)-α-D-glucopyranosyl-(1,2)-β-D-fructofuranoside), in which, instead of the glucoside group of the saccharose, a galactopyranosyl-(1,6)-α-D-glucopyranosyl group is bonded to the β-D-fructofuranosyl group in 1,2 linkage. In the description of the present invention, all references to saccharose and saccharose analogues also hold for raffinose and its analogues.

Possible acceptor molecules include (poly-) hydroxyl compounds, e.g. saccharides, and thiol compounds, as well as peptides, proteins, or drugs. Both specific embodiments of the method according to the present invention can be used, through multiple application to an acceptor molecule, to transfer a plurality of identical or different ketofuranosyl groups or aldopyranoside groups one after the other, so that oligosaccharides are produced having different molecular masses.

In a first specific embodiment, the method is used to transfer the ketofuranosyl group onto acceptor molecules that have at least one hydroxyl group as an acceptor, but that need not necessarily be pure glycoside compounds. Examples of substrate molecules that are glycosides include β-D-ketofuranosyl-D-aldopyranosides and derivates of β-D-fructofuranosyl compounds. In this first specific embodiment, according to the present invention the derivatized fructosyl group is transferred onto the acceptor molecule. Such a reaction is catalyzed by ketofuranosyl transferases, as well as by fructosyl transferases, e.g. fructosyl transferases from Bacillus subtilis, or by a glycosyl transferase.

According to the present invention, it is preferred that the β-D-ketofuranosyl-D-aldopyranoside compound used as a substrate, in which the ketofuranosyl group is an isomer of fructose in the form of a derivatized fructofuranosyl compound, is produced enzymatically from saccharose. This can be achieved by conversion of saccharose having the desired fructose derivate, so that a saccharose analogue results in which the original fructosyl group is replaced by a fructosyl derivate. For the catalysis, a glycosyl transferase, e.g. a fructosyl transferase, can be used.

For the purposes of the present invention, the term “derivatized fructofuranosyl compound” or “fructosyl derivate” is understood to refer to substituents that are able to replace the fructosyl group of the saccharose, preferably in enzymatically catalyzed reaction from saccharose. Examples of derivatized fructofuranosyl groups include other ketofuranosyls, e.g. fructosyl derivates and psicosyl, sorbosyl, and tagatosyl groups, as well as derivates thereof.

In a second specific embodiment, the present invention relates to the transfer of the D-aldopyranoside group from β-D-ketofuranosyl-α-aldopyranosides onto an acceptor molecule. Possible acceptor molecules include hydroxyl and thiol compounds, e.g. saccharides. The transfer of the aldopyranoside group is catalyzed by a specific glycosyl transferase. Examples of specific glycosyl transferases include those that specifically transfer a mannopyranosyl, galactopyranosyl, fucopyranosyl, or rhamnopyranosyl group that is bound in α 1-2 to a ketofuranosyl group (e.g. to a fructofuranosyl group), or a derivatized glycopyranosyl group, onto acceptor molecules. As acceptor molecules, it is possible to use carbohydrates, peptides, proteins, alcohols, drugs, and natural materials that carry a hydroxyl group and/or a thiol group.

In the second specific embodiment of the present invention, it is preferred that the β-D-ketofuranosyl-D-aldopyranoside compound be produced enzymatically from saccharose, namely through conversion of saccharose with the desired aldopyranose under catalysis using, for example, a fructosyl transferase (see for example FIG. 3). In this way, the glucoside group of the saccharose can be replaced by another aldopyranoside group that, in a following enzymatic catalysis step, is transferred onto the acceptor molecule (FIG. 4).

In the second specific embodiment, the synthesis method according to the present invention can be used to produce specific disaccharides and longer-chain oligo- or polysaccharides in which an acceptor molecule, e.g. a monosaccharide or disaccharide, is prolonged in each case by the aldopyranoside group of a β-D-ketofuranosyl-α-D-aldopyranoside. Here, the aldopyranoside group can be the same or different in successive reactions, so that an oligosaccharide chain is constructed from identical and/or differing aldopyranoside components.

Correspondingly, the synthesis method according to the present invention can be used to produce specific disaccharides and longer-chain oligosaccharides in which β-D-ketofuranoside groups or aldopyranoside groups are transferred onto an acceptor molecule. In successive reactions, identical or different derivatized ketofuranoside and/or aldopyranoside groups can be transferred in this way, so that an oligosaccharide chain is constructed from identical and/or differing ketofuranosyl and/or aldopyranosyl groups.

Oligosaccharides according to the present invention, in particular fructooligosaccharides, are suitable in particular for use as food supplements having a prebiotic effect. This is because, differing from saccharose, maltose, and other glycosides, the fructosyloligosaccharides are not easily hydrolyzed in the acidic environment of the stomach or enzymatically, but rather can at least to a significant extent move from the small intestine into the large intestine. Here they can deploy their prebiotic effect, because there they are metabolized by the probiotically effective bifidobacteria, such as for example through breakdown to form short-chain fatty acids. In this way, the oligosaccharides according to the present invention promote the survival and growth of the bifidobacterium, which supports and maintains important physiological functions of the digestive system. In addition, it can be shown that the absorption of oligosaccharides according to the present invention in the human digestive system promotes the absorption of minerals, for example calcium ions, iron (III) ions, and zinc ions, which help to prevent osteoporosis. In addition, fructooligosaccharides in particular according to the present invention have an effect on metabolism of fats, and result in a reduction in the plasma mirror of cholesterol, as well as to an increase in the HDL/LDL cholesterol ratio, resulting in a reduction in the risk of arteriosclerotic vascular diseases.

In sum, therefore, the prebiotic effect of oligosaccharides according to the present invention, in particular fructooligosaccharides, which preferably have an aldopyranose as the head group that is not glucose, results, directly or by promoting the growth of probiotic bifidobacteria, in a health-promoting effect, in particular because the oligosaccharides according to the present invention are essentially not digestible. As a head group, in particular galactose, mannose, fucose, xylulose, respectively in the D- or L-configuration, L-glucose, L-rhamnose, and in particular galactosylmelibiose are preferred.

### DETAILED DESCRIPTION OF THE PRESENT INVENTION

In the first specific embodiment of the present invention, a method and a combination of materials for carrying out this method are provided with which a ketafuranosyl group, namely the fructosyl group or a derivatized fructosyl group, from a derivatized β-D-ketofuranosyl-α-D-aldopyranoside as a saccharose analogue, is transferred onto an acceptor molecule. Possible acceptor molecules can be compounds containing hydroxyl groups or containing thiol groups, for example saccharides, including the saccharose analogue itself, peptides, drugs, synthetic or natural polymers. For the transferase reaction, the corresponding ketofuranosyl derivates of the aldopyranoside, preferably of the glucopyranoside, are then to be used. To the extent that the specificity of the preferably used fructosyl transferase is not suitable for the transfer of a ketofuranosyl group, i.e., of a derivatized fructosyl group, suitable specific ketofuranosyl transferases can be produced through mutagenesis and screening and identified, as is analogously described below for the production and identification of glycosyl transferases that are specific for aldopyranosides. Here, as a substrate, instead of the respective aldopyranoside derivate, the respective specific ketofuranosyl derivate is to be used.

Correspondingly, the first specific embodiment also relates to oligosaccharides or polysaccharides that can be obtained by transferring the ketofuranosyl group. If saccharose analogues are used for the transferase reaction whose fructosyl group is derivatized, an oligomer is obtained that has derivatized fructosyl components. Compounds of the first specific embodiment according to the present invention are therefore oligo-/poly-(ketofuranosyl) compounds in which the ketofuranosyl groups are fructosyls and/or derivatized fructosyls.

If saccharose analogues whose aldopyranoside group is not the glucose group are converted in the transferase reaction, suitable glycosyl transferases can be used to transfer the fructosyl group, so that an oligo- or polyfructoside having the respective aldopyranoside group is obtained as a head group provided with fructosyl groups. This latter variant is suitable for producing oligo- or polyfructosyl aldopyranoside compounds, also called pyranosyloligofructoside or pyranosylpolyfructoside, catalyzed by a fructosyl transferase. In this method, enzyme activities outside the desired fructosyl transferase, differing from a polyfructosylation from saccharose, are not disturbing, such as for example a dextrane sucrase. Here, the present invention exploits the circumstance that the enzymatically more active dextrane sucrase does not polymerize the derivatized, or formally exchanged for glucose, aldopyranoside group, while the enzymatically less active fructosyl transferase transfers the fructosyl group from the saccharose analogue onto an acceptor without significant limitation. A suitable fructosyl transferase is known from Leuconostoc mesenteroides or Bacillus subtilis. Therefore, the compounds of the first specific embodiment according to the present invention also include oligo-/polyfructosyl compounds that can be obtained through conversion of a fructosyl aldopyranoside, in which the aldopyranoside is not glucose and is released.

Preferred pyranosyloligofructosides or pyranosylpolyfructosides contain in the range from 2 to 10⁶, preferably 2 to 100, particularly preferably 5 to 20 or 10 fructosyl units. The fructosyl units are preferably linked to one another glycosidically in C2-C6 and/or C2-C1. The C2-C1 linkage can be catalyzed for example by an inulosucrase. The pyranoside group is preferably glycosidically bound to a fructose unit at the end position in β-1,2.

In a second, preferred specific embodiment of the present invention, a method is provided for the synthesis of oligosaccharides or for the glycosylation of acceptor molecules containing hydroxyl groups and/or containing thiol groups, by which an aldopyranoside group is transferred from a β-D-ketofuranosyl-α-D-aldopyranoside compound. The preferred β-D-ketofuranosyl-α-D-aldopyranoside is the saccharose, preferably a saccharose derivate, that contains as aldopyranoside, instead of the glucose group, the group of another aldopyranose, or a glycoside derivate.

Correspondingly, the second specific embodiment also relates to oligo- or polysaccharides that can be obtained by transferring the aldopyranoside group. If saccharose analogues are used for the transferase reaction whose aldopyranoside group is a derivatized glucoside group or an aldopyranoside group other than glucose, an oligomer or polymer is obtained having the derivatized or other aldopyranoside components as glucose. Compounds of the second specific embodiment according to the present invention are therefore oligo-/poly-(aldopyranosyl) compounds in which the aldopyranosyl group is derivatized glucosyl or some aldopyranosyl other than glucosyl.

For the purposes of the present invention, derivates of saccharose in which the fructosyl group is formally replaced by a derivate of the fructosyl group or by another ketofuranoside group, and/or the glucoside group is formally replaced by a derivate of the glucoside group or by another aldopyranoside group, are designated saccharose analogues.

The method according to the present invention is based on putting into practice the finding that the binding energy of the glycosidic binding of saccharose (−23 kJ/mol) is sufficient to transfer, in a kinetically controlled synthesis, the ketofuranosyl group or the aldopyranoside group onto an acceptor molecule, even if in the saccharose analogues only one of these two groups has been replaced through the formal exchange of the ketofuranosyl group for a fructose isomer or a fructosyl derivate, and/or the glucopyranoside group has been replaced by another aldopyranoside group.

Therefore, for this transferase reaction the duration of the reaction and the concentrations of the educts and products prevailing during the transferase reaction are to be set in such a way that the transferase reaction is essentially terminated after the highest concentration of product is achieved. For the first and second specific embodiment, these kinetically controlled transfer reactions can be carried out and regulated in suitable reactor types, for example through the use of immobilized enzymes and suitable limited contact times, and/or by controlling the concentrations of educts and product (Böker et al., Biotech. Bioeng. 43, 856-864, (1994)).

The glycosyl transferase that is to be used for the method according to the present invention, which is specific for a ketofuranoside group or for an aldopyranoside group, or for derivates of these glycoside groups, i.e. that can transfer a desired ketofuranoside group or an aldopyranoside group from a β-D-ketofuranosyl-α-D-aldopyranoside compound, e.g. from a saccharose analogue, onto an acceptor molecule, can be identified in the screening method. Thus, known enzymes, e.g. fructosyl transferase or dextrane sucrase, can be identified for their specificity for a particular ketofuranoside group or aldopyranoside group in a β-D-ketofuranosyl-α-D-aldopyranoside in that the enzyme converts the specific β-D-ketofuranosyl-α-D-aldopyranoside in vitro. Given an enzyme having the sought specificity, the conversion results in the transfer of the ketofuranoside group or of the aldopyranoside group onto an acceptor molecule, for example for the oligomerization with simultaneous release of the aldopyranoside group or ketofuranosyl group of the saccharose analogue used as a substrate. The activity of a specific enzyme can therefore be determined for example through colorimetric and/or photometric detection of the hydrolysis product. Given the release of fructose after the transfer of an aldopyranoside group from a β-D-fructofuranosyl-α-D-aldopyranoside, the fructose can be spectrophotometrically determined as a free hydrolysis product after isomerization through glucose isomerase to glucose, phosphorylation by means of hexokinase, and oxidation through glucose-6-phosphate dehydrogenase, with simultaneous reduction of NADP to NADPH. This test system is applicable to various aldopyranoside derivates, i.e. for example saccharose analogues in which the glucoside group is replaced for example by another aldopyranoside or by a derivate of the glucoside group.

In another preferred embodiment of the present invention, the β-D-ketofuranosyl-α-D-aldopyranoside is produced enzymatically. In the second specific embodiment of the present invention, a desired β-D-ketofuranosyl-α-D-aldopyranoside is produced, preferably starting from saccharose, as β-D-fructosyl-α-D-aldopyranoside in which the glucoside group of the saccharose has been exchanged for the desired aldoside group. This can be catalyzed by a fructosyl transferase, e.g. the fructosyl transferase from Bacillus subtilis (NCIMB 11871). Such β-D-fructosyl-α-D-aldopyranosides can also be designated saccharose analogues, because the saccharide binding of the saccharose remains, although the original glucoside group is replaced by a new pyranoside group.

For the first specific embodiment of the present invention, it is preferred to enzymatically produce saccharose analogues in which the fructosyl group is formally exchanged for a fructosyl derivate or for another ketofuranosyl group. This reaction can be catalyzed by a glucosyl transferase.

Saccharose analogues that are to be used according to the present invention are for example compounds in which the fructosyl group of the saccharose is replaced by a group that is selected from a ribulose, lyxose, xylulose, derivatized fructose, ribulose, psicose, sorbose, or tagatose group, or some other pentose, hexose, or heptose that is glycosidically bound to the Cl of the glycoside group, in each case in a D or L configuration, or substituted derivates of the above-named groups.

Examples of saccharose analogues that can be used in the second specific embodiment of the present invention are β-D-fructosyl compounds that have, at the C2 of the fructosyl group, a ribose, arabinose, xylose, lyxose, allose, altrose, galactose, mannose, gulose, idose, talose, fucose, 2-N-acetylglucosamine, 2-N-acetylgalactosamine, 2-deoxyglucose, 3-deoxyglucose, 4-deoxyglucose, 6-deoxyglucose, 2-deoxygalactose, 3-deoxygalactose, 3-ketoglucose, 4-ketoglucose, sialinic acid, or N-acetyl-neuraminic acid group, each in the D or L configuration, L-glucose, or some other glycosidically bound pentose, hexose, heptose, or substituted derivates of the above-named groups, e.g. a derivatized glucose group.

As acceptor molecules, it is possible to select alcoholic hydroxyl groups of carbohydrates, steroids, terpenes, polyketides, hydroxy amino acids, hydroxy nitriles, other metabolic products of microorganisms, diglycerides, ceramides, enolic and/or phenolic hydroxy groups of phenols, flavonoids, as well as mercapto groups and amide groups, for example of asparagine, threonine, serine, cystein, for example as a component of peptides, purines, pyrimidines, benzimidazol or nicotinic acid amide.

According to the present invention, in a preferred embodiment it is provided that the saccharose analogues that are used to produce pyranosyloligofructosides or pyranosylpolyfructosides that are produced through enzyme-catalyzed conversion of the pyranose with saccharose, corresponding to the other specific embodiments of the present invention.

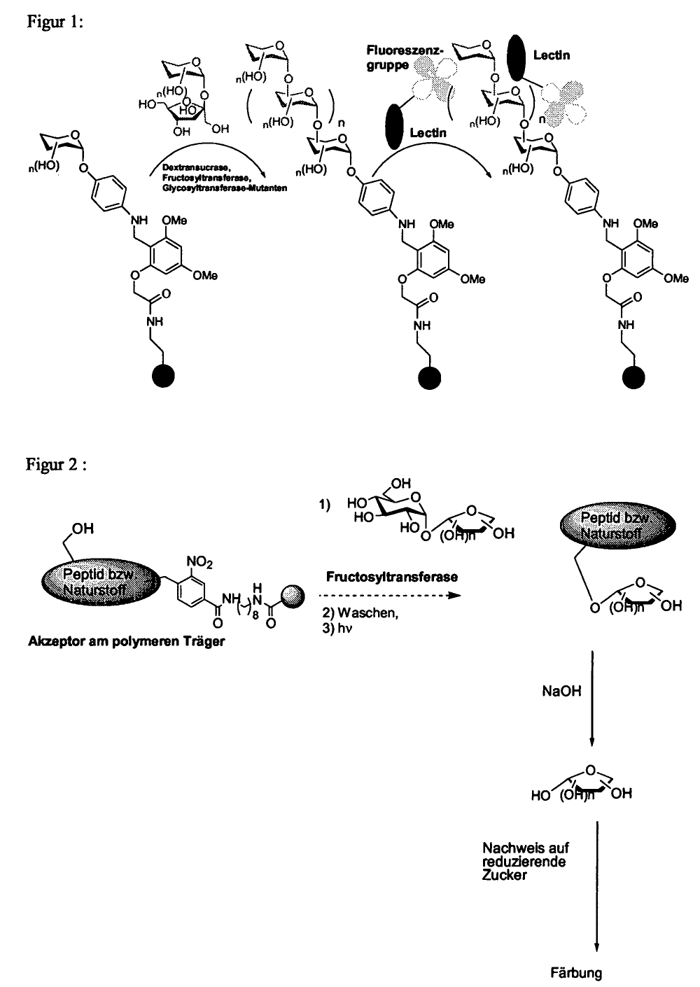

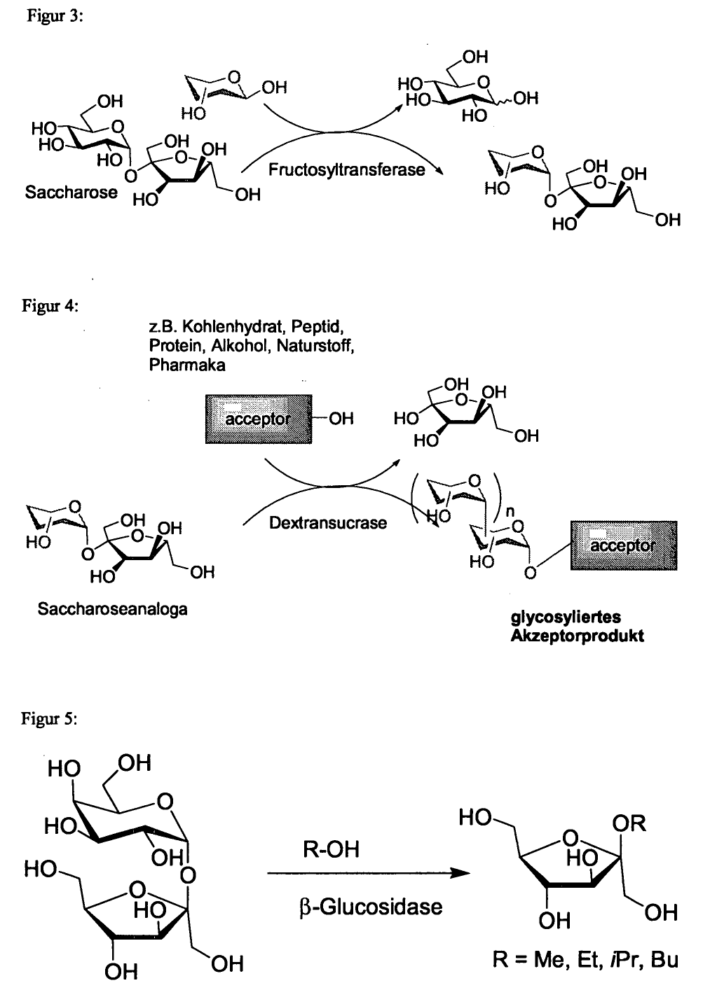

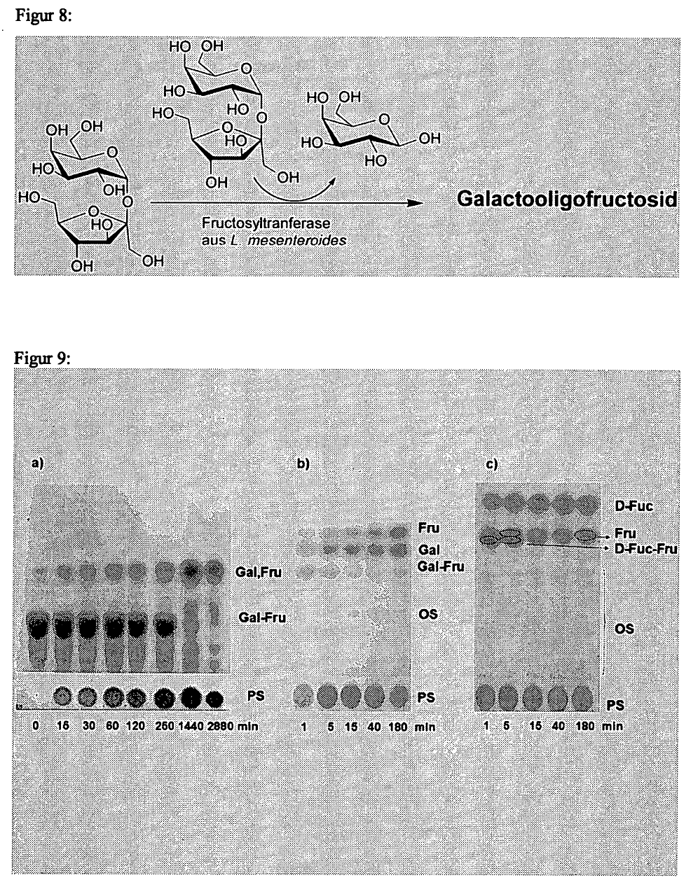

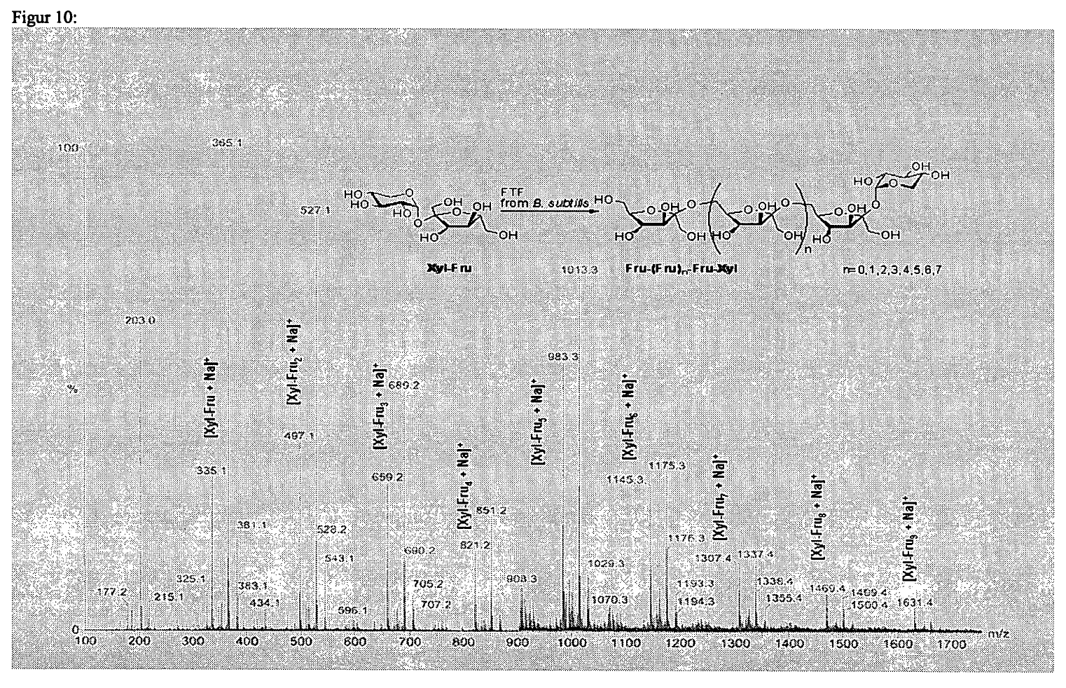

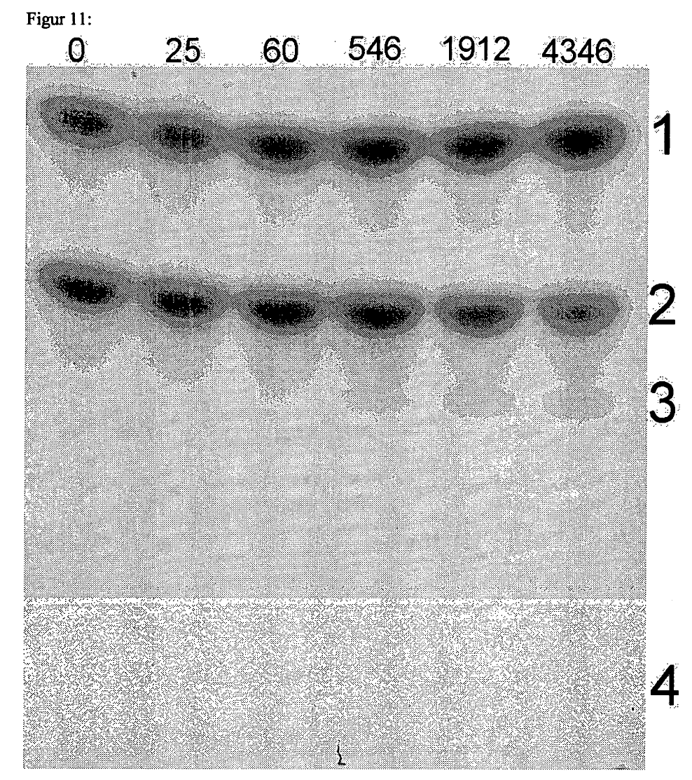

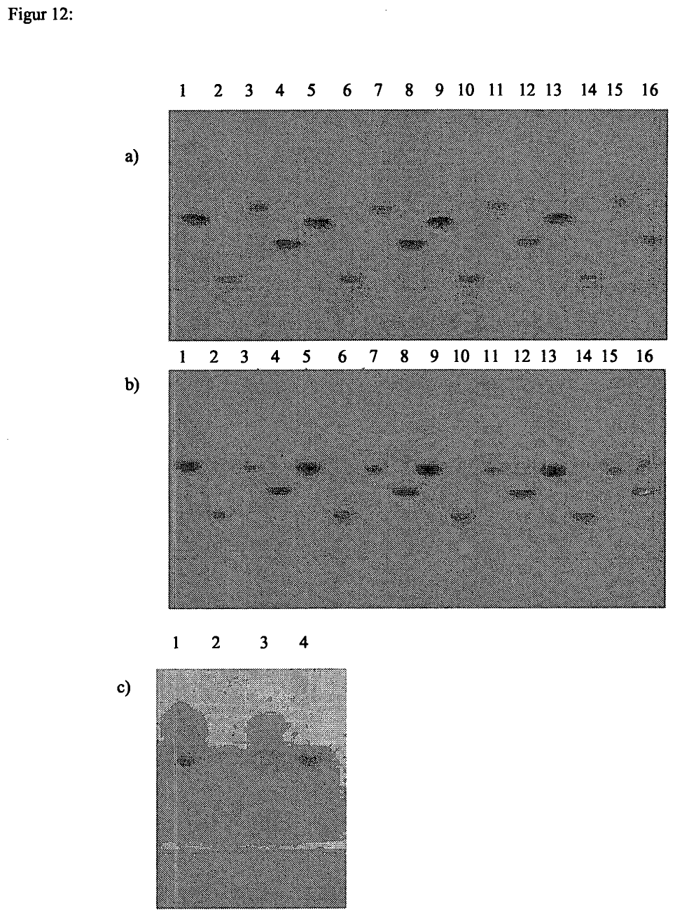

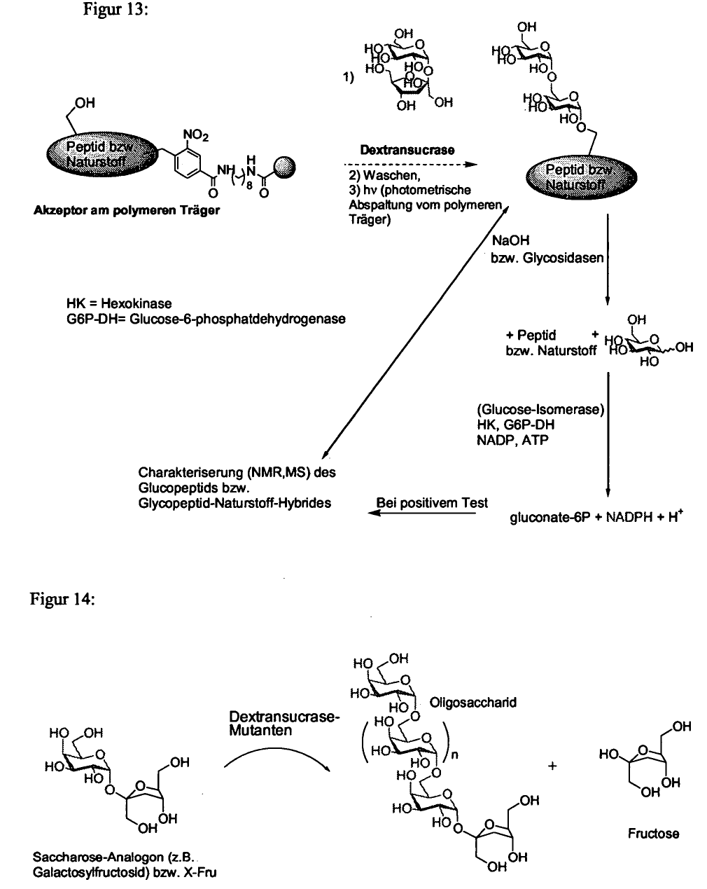

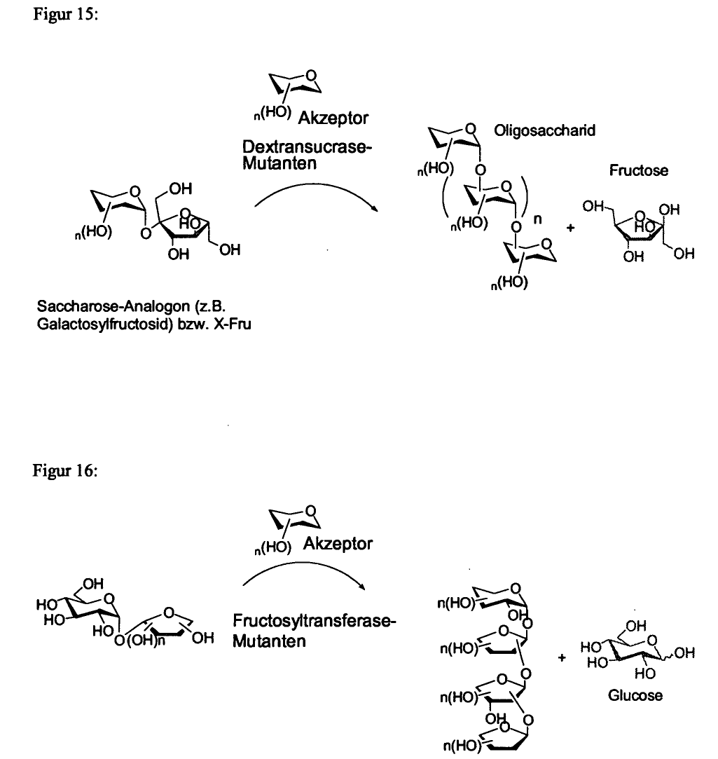

The present invention is now described in detail on the basis of examples with reference to the Figures, in which
FIG. 1 shows a schematic representation of the screening of suitable aldopyranoside transferases,
FIG. 2 shows a schematic representation of the screening of suitable ketofuranoside transferases,
FIG. 3 is a schematic representation of the preferred synthesis of the saccharose analogues,
FIG. 4 is a schematic representation of the glycosylation of an acceptor molecule having a glycoside that was first transformed into a saccharose analogue,
FIG. 5 is a schematic representation of the transfer of a fructosyl group from an aldosylfructoside onto an alcohol,
FIG. 6 is a schematic representation of the transfer of a fructosyl group from an aldosylfructoside onto an amino acid derivate,
FIG. 7 is a schematic representation of the synthesis of a xylosyloligofructosside from Xyl-Fru, with a fructosyl transferase from Bacillus subtilis,
FIG. 8 is the schematic representation of the synthesis of a galactooligofructoside from the saccharose analogue Gal-Fru with a fructosyl transferase from Leuconostoc mesenteroides,
FIG. 9 shows thin-layer chromatograms concerning the curve of the synthesis to form polysaccharides (PS) according to the present invention, among a) Gal-Fru-(Fru)ₙ, b) Gal-Fru-(Fru)ₙ [sic], and c) Fuc-Fru-(Fru)ₙ,
FIG. 10 shows the ESI-MS spectrum of a transfructosylation reaction according to the present invention for Xyl-Fru-(Fru)ₙ, and its reaction schema,
FIG. 11 shows a thin-layer chromatogram of the synthesis of mannosyl oligofructoside from D-Man-Fru,
FIG. 12 shows thin-layer chromatograms of the course of the synthesis of polysaccharides according to the present invention,
FIG. 13 is a schematic representation of the glycosylation of an acceptor molecule immobilized at the polymeric carrier, with subsequent detection of the synthesized oligosaccharide,
FIG. 14 is a schematic representation of the transfer of a furanoside group from a saccharose analogue (galactosylfructoside) onto a saccharide in order to produce an oligosaccharide,
FIG. 15 is a schematic representation of the oligomerization or polymerization of pyranoside groups originating from a saccharose analogue, and
FIG. 16 is a schematic representation of the oligomerization of furanoside groups originating from a saccharose analogue.

### EXAMPLE 1

### Production and Identification of a Specific Glycosyl Transferase

For the present invention, it is preferred to use a specific glycosyl transferase for the transfer of the ketofuranosyl group or of the aldopyranoside group from a saccharose analogue onto an acceptor molecule. For the transfer of aldopyranoside groups from saccharose analogues, first mutations of dextrane sucrase are produced. For the mutagenesis, the genes of the glycosyl transferase GtfR from Streptococcus oralis ATTC 10557, DsrS from Streptococcus mesenteroides (Fujiwara et al., 2000), GtfB and GtfC (Streptococcus mutans) were coupled with inducible promoters. As inducible promoters, an IPTG-dependent promoter, alternatively a promoter capable of being regulated with arabinose or by dihydrotetracycline, was used.

The glycosyl transferase genes were subjected to a statistical mutagenesis, preferably using region-specific PCR that related to individual segments of the genes, for example the domains involved in substrate binding. In this way, individual segments of the glycosyl transferase genes were mutated that subsequently formed a gene library of mutated gene sequences and were combinatorially linked to one another to form new glycosyl transferase genes. Alternatively, the libraries of gene segments were used to replace the corresponding segment in the wild-type sequence. For the screening of the obtained substrate specificities of the mutated glycosyl transferase genes, these were expressed in E. coli (BL21).

The screening took place through the addition of the respective saccharose analogue and colorimetric or spectrophotometric determination of the released reducing sugar. This is because, given the presence of a specificity for the fructosyl derivate group or aldopyranoside group of a saccharose analogue, the non-transferred saccharide group is released as a by-product. This released saccharide group is reducing and can be spectrophotometrically determined using standard methods.

Through the screening of mutated glycosyl transferase genes, specific transferases were found for the saccharose analogues that are here generally and concretely designated, and in particular for the following saccharose analogues having high specific transferase activity (here only the saccharide group formally replaced in relation to the saccharose is indicated): α-D-mannoside, α-D-galactoside, α-D-xyloside (spectrophotometric measurement of released fructose or glucose).

For a more precise characterization of the obtained glycosyl transferase mutations, these were subjected to a secondary screening in which transferases were incubated with an acceptor molecule and the specific substrate. The formed products are subsequently analyzed in thin-layer chromatography, using HPLC-MS and NMR.

For the identification of glycosyl transferases that synthesize oligosaccharides that bind lectin, a screening method can be used in which a lectin for which a specifically binding oligosaccharide is sought is used to identify the oligosaccharide synthesized at a solid phase. For a simple detection method, the lectin is coupled to a reporter group, e.g. a fluorescing molecule. For the solid-phase-bound synthesis of the oligosaccharide that is synthesized from the saccharose analogue by the glycosyl transferase, an acceptor molecule is used that is coupled to a solid phase and that has a free hydroxyl group. This can for example be the phenolic hydroxyl group as shown schematically for the screening method in FIG. 1.

For the identification of suitable glycosyl transferases, known enzymes or mutagenesis products thereof are screened, such as dextrane sucrases, fructosyl transferases, or mutations of glycosyl transferases. FIG. 1 schematically shows a screening method in which the specificity of the transferase is identified in that a lectin having specificity for the desired oligosaccharide is used to identify the oligosaccharide that is synthesized from the saccharose analogues provided as co-substrates.

For a simple analysis, the acceptor is solid-phase-coupled, so that the synthesized oligosaccharide is also bound to the solid phase and can easily be separated from the other components of the reaction. After reaction with the lectin, the specific binding can be confirmed by detection of a group coupled to the lectin, e.g. a fluorescing molecule. In the case shown in FIG. 1, saccharose analogues are used in which the glucoside group is exchanged for another component, for example another aldopyranoside group or a derivatized glucoside group.

The screening method of FIG. 1 can also be carried out with saccharose analogues that have, instead of the fructosyl group, a derivatized fructosyl group or another ketofuranoside group, in order to identify a transferase that specifically transfers the respective ketofuranoside group.

An example of screening for a fructosyl transferase having specificity for the transfer of a ketofuranoside group from a saccharose analogue is shown in FIG. 2. The acceptor molecule is likewise immobilized through binding to a polymeric carrier, and the specificity of the fructosyl transferase is analyzed in that, given the use of a saccharose analogue in which the fructosyl group is exchanged for another ketofuranoside group or a derivate of the fructosyl group, reducing sugars are determined through subsequent hydrolysis of the oligosaccharide synthesized on the acceptor molecule.

Using saccharose analogues that have, instead of the glucoside group, another aldopyranose or a derivate of the glucoside group, this screening method can also be used for the screening of glycosyl transferases that is [sic] specific for the respective group of the saccharose analogue that replaces the original glucoside group.

For the example of the glycosyl transferase R from ATCC 10557 (contained as sequence ID no. 5) (Fujiwara et al., Infect. Immun. 68: 2475-2483 (2000)), available under AB025228; BAA 95201.1 (EMBL), it can be shown that the substrate spectrum of the enzyme designated as the wild type as glucosyl transferase can be modified through individual exchanges, insertions, or deletions of amino acids. Using the above-described method for locus-directed mutagenesis, variants of the wild-type sequence are produced that have different substrate specificities and are summarized in Table 1.

TABLE 1

New glycosyl transferases based on glucosyl
transferase R from ATCC 10557

| | Partial | |
|---|---|---|
| | sequence, | |
| | amino acids | |
| Enzyme | nos. 620-634 | Enzymatic properties |
| Wild type | YIFVRAHDSEVQTVI | Glucosyl transferase; |
| (seq. ID | | polymer units are α-1,6- |
| no. 4) | | linked |
| Variant 1 | YIFVRAHDDEVQTVI | No polymer formation; |
| (seq. ID | | strong acceptor binding; |
| no. 1) | | different substrate |
| | | spectrum |
| Variant 2 | YIFVRAHDREVQTVI | No polymer formation; |
| (seq. ID | | strong acceptor binding; |
| no. 2) | | different substrate |
| | | spectrum |
| Variant 3 | YIFVRAHDSEIQTVI | Polymer having different |
| (seq. ID | | properties |
| no. 3) | | |

The numbering of the amino acids relates to the published wild-type sequence named above; the introduced mutations are underlined.

### EXAMPLE 2

### Synthesis of a Disaccharide Having Immobilized Glycosyl Transferase

For the immobilization of glycosyl transferases, known carriers, e.g. Eupergit-C (Röhm & Haas), can be used. Another suitable method for immobilization is encapsulation in alginate, which is a method known to those skilled in the art. Enzymes immobilized in this way can be used in continuous flow reactors or in batch reactors, as is generally known in the prior art (Reischwitz et al., Enz. Microb. Technol. 19, 518-524 (1996)).

### EXAMPLE 3

### Synthesis of Saccharose Analogues

For the enzymatic synthesis of β-D-fructofuranosyl-α-D-aldopyranosides that are to be used according to the present invention, the glucoside group of the saccharose is replaced by the desired aldopyranoside. This reaction is carried out using the fructosyl transferase from Bacillus subtilis NCIB 11871, and it can be shown that given sugar concentrations in the reaction solution of from 10 to 400 g/L, preferably 100-300 g/L, saccharose analogues can be produced. The products can be separated using ion exchangers.

The schematic sequence of the synthesis of saccharose analogues is shown in FIG. 3 for the example of the replacement of the glucosyl group by a freely selectable aldopyranoside group. Here, saccharose with an aldopyranose as a co-substrate, catalyzed by fructosyl transferase, is converted to aldopyranoside-1,2-β-D-fructosylfuranoside, with the release of glucose.

In the following Tables 1 and 2, the co-substrates used for the conversion of saccharose and the saccharose analogues obtained with the fructosyl transferase reaction are indicated as disaccharides or trisaccharides:

TABLE 1
Disaccharide-saccharose analogues synthesized from saccharose

| Co-substrate | Saccharose analogue | Product yields [g/L] |
|---|---|---|
| 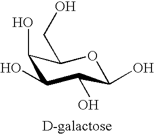 | 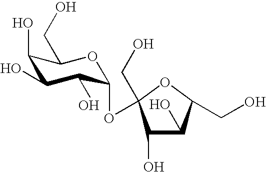 | 256 |
| 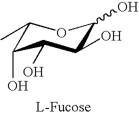 | 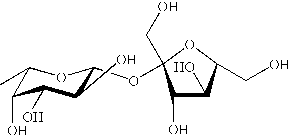 | 53 |
| 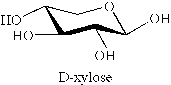 | 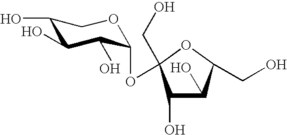 | 240 |
| 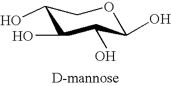 | 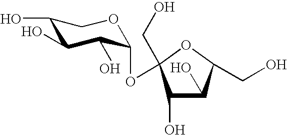 | 10 |
| 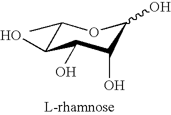 | 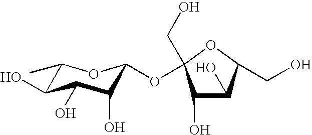 | 3 |
| 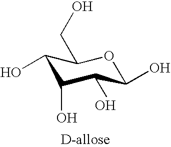 | 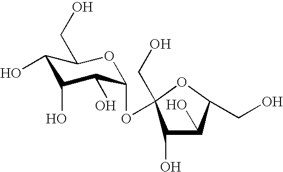 | 3 |
| 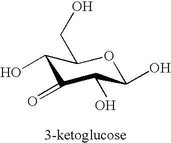 | 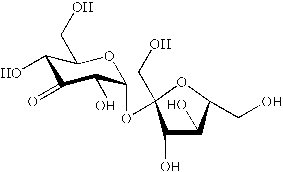 | |

TABLE 2
Trisaccharide-saccharose analogues synthesized from saccharose

| | | Product |
|---|---|---|
| Co-substrate | Saccharose analogue | yields [g/L] |
| 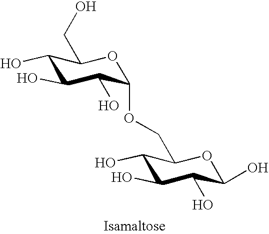 | 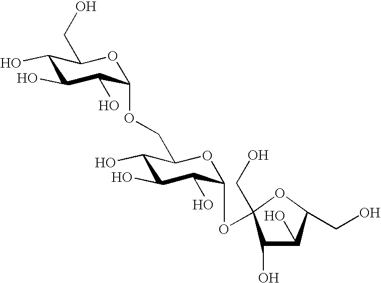 | 218 |
| 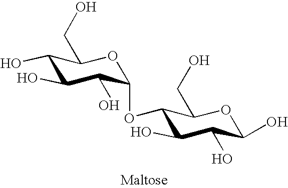 | 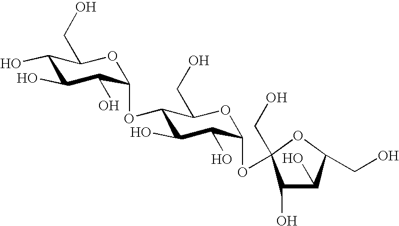 | 261 |
| 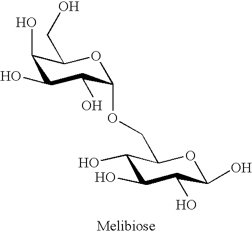 | 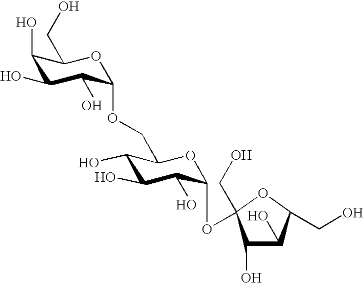 | 157 |
| 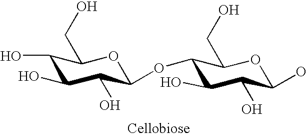 | 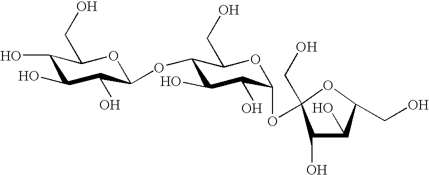 | 223 |
| 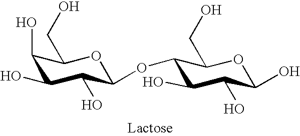 | 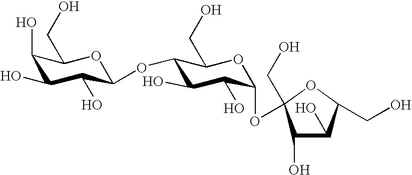 | 72 |

### EXAMPLE 4

### Glycosylation of an Acceptor by Transferring the Pyranoside Group from a Saccharose Analog

FIG. 4 schematically shows how, according to the present invention, the aldopyranoside group of a saccharose analogue is transferred onto an acceptor, which here has a hydroxyl group. With the aid of a known glycosyl transferase, or a glycosyl transferase modified through mutagenesis, the aldopyranoside group is transferred onto the hydroxyl group of the acceptor molecule, with an accompanying release of fructose. As a result, as a product an acceptor molecule is obtained that is glycosylated one or more times with the aldopyranoside group.

### EXAMPLE 5

### Fructosylation Through Transfer of the Fructoside Group from a Saccharose Analogue onto an Acceptor Containing a Hydroxyl Group

The production of a fructosyl derivate according to the present invention is shown schematically in FIG. 5 for the example of the transfer of the fructose group from the saccharose analogue galactosyl fructoside onto alcohols. With catalysis of the β-glucosidase, the fructoside group is transferred onto the hydroxyl group of the co-substrate alcohol, so that a fructosylized alcohol is obtained. The galactoside group is released as galactose. Through continuation of the reaction, an oligo- or polyfructosylation of the acceptor can be achieved, one additional fructosyl group being transferred onto the acceptor in each case.

### EXAMPLE 6

### Fructosylation by Transferring the Fructoside Group from a Saccharose Analogue onto an Amino Acid

FIG. 6 schematically shows the transferring of the fructosyl group from a saccharose analogue, here a galactosylfructoside, onto an amino acid. The amino acid is derivatized, namely serine, whose carboxyl and amine groups each have a protective group. This protected serine is representative of amino acids that are bound in a peptide and that have reactive acceptor groups that are suitable for fructosylation. Besides the indicated hydroxyl group, these can be thiol groups and amine groups. With catalysis of the β-glucosidase, the fructosyl group is transferred onto the hydroxyl group of the serine, so that a fructosylated serine derivate, representative of fructosylated peptides, is obtained.

### EXAMPLE 7

### Synthesis of Pyranosyloligofructosides

The production of a pyranosyloligofructoside or pyranosylpolyfructoside according to the present invention is shown schematically in FIG. 7 for the example of the xylosyl-di- or -polyfructoside having four fructosyl units, through conversion of xylosylfructoside with fructosyl transferase from Bacillus subtilis. The fructosyl group of the saccharose analogue xylosylfructoside is transferred by the fructosyl transferase onto xylosylfructoside, so that, inter alia, the indicated xylosylfructoside is obtained, and, as the transferase reaction is continued, a pentaglycoside is obtained having for example the depicted structure of four fructosyl groups and one xylosyl. Through continuation of the reaction, longer fructosyl chains can be obtained having at least 5 to 100 fructosyl units. The bonds of the fructosyl units are C2-C6, and partly also C2-C1. The end-position pyranoside group, here xylosyl, is, corresponding to the saccharose analogue, bound in the α-position at C1 to the C2 of the next fructosyl unit in the β-position.

FIG. 8 schematically shows the transfer of the fructosyl group from Gal-Fru with the aid of the fructosyl transferase from Leuconostoc mesenteroides. Corresponding to the synthesis of the xylosyloligofructoside, a galacto-oligo- or -polyfructoside is obtained.

For the synthesis of oligosaccharides according to the present invention, fructosyl aldopyranosides were used as a substrate that were obtained through the conversion of saccharose with the aldopyranose replacing the respective glucose group. For catalysis, a fructosyl transferase was used.

In addition to D-Gal-Fru, D-Fuc-Fru was also used in order to produce, using the fructosyl transferase from L. mesenteroides (FTF-a) or B. subtilis (NCIMB 11871, FTF-2), D-Gal-Fru-(Fru)₂₀₋₁₀₀ and D-Gal-Fru-(Fru)_{>100} or D-Fuc-Fru-(Fru)₂₀₋₁₀₀ and D-Fuc-Fru-(Fru)_{>100}. The course of the synthesis is shown in FIG. 9 on the basis of thin-layer chromatograms, in which a) shows the course of the synthesis with FTF-1 (137 U/L), and b) shows the synthesis course with FTF-2 (2860 U/L) of the conversion of D-Gal-Fru, and c) shows the synthesis course with FTF-2 (2860 U/L) of the conversion of D-Fuc-Fru. In each case, it can be seen that over the time shown the polysaccharide (PS) named above is synthesized, such that oligosaccharides (OS) cannot be separated and longer-chain PS remain at the application point (inserted into the chromatograms at bottom).

As FIG. 10 shows schematically, Xyl-Fru-(Fru)₁₋₅₀ was also synthesized from Xyl-Fru with FTF-2. FIG. 10 shows the ESI-MS spectrum of Xyl-Fru-(Fru)₁₋₅₀, in which the signals 335.1, 497.1, 659.2, 821.2, 983.3, 1145.3, 1307.4, 1469.4, 1631.4 indicate ([M+Na]⁺) oligosaccharides of the type Xyl-(Fru)ₙ, where n is 1-9. Higher molecular weights than those indicated in FIG. 10 were estimated in thin-layer chromatograms.

The particular advantage of the synthesis according to the present invention can be seen in that the addition of dextrane sucrase does not result in the production of dextrane. This is due to the fact that the saccharose analogues that are used according to the present invention, whose glucoside group is derivatized or exchanged for another group, for example another hexose, is not a substrate for dextrane sucrase. Therefore, it is possible to produce oligo- or polyfructosides from saccharose analogues without secondary reactions causing the occurrence of dextrane or a polymer of the aldopyranoside groups as contaminants.

Another example for the transfer of the fructosyl group from a saccharose analogue is the transfer of the fructosyl group from D-Man-Fru with FTF-2. For the production of a mannosyl oligofructoside according to the present invention of the type (D-Man-Fru-(Fru)₁₋₈), D-Man-Fru was incubated with FTF-2, this saccharose analogue acting both as acceptor and also as substrate for the transfer of the fructosyl group. In order to suppress the secondary reaction, which can occur as hydrolysis of the saccharose analogue, in principle one of the hydrolysis products can be added, preferably the aldopyranoside, here D-mannose. Under otherwise identical reaction conditions, the yield can be increased by this addition to the reaction mixture.

The result of the conversion of D-Man-Fru for the fructosylation thereof with the same saccharose analogue as substrate is shown in the thin-layer chromatogram in FIG. 11, where reference character 1 identifies the D-mannose, 2 identifies the saccharose analogue, and 3 is the D-mannosyl-oligofructoside (D-Man-Fru-(Fru)₁₋₈). The indications for the individual tracks of the thin-layer chromatography state the reaction duration in minutes. The segment designated 4 indicates that even after a reaction duration of three days, saccharose analogue can still be detected as a substrate.

These examples also show that the binding energy of the saccharose analogues that are to be used according to the present invention is sufficient in each case to transfer one of the two glycoside groups with accompanying release of the other glycoside group.

### EXAMPLE 8

### Synthesis of Oligosaccharides According to the Present Invention from L-aldopyranosyl-polyfructoside

As an example of polyfructosides that carry as the main group an aldopyranose in the L configuration, L-glucose, L-galactose, L-xylulose and L-glucose fructoside (comparison example) are each produced with catalysis by fructosyl transferase (FTF-2), through conversion of the respective L-aldopyranose with saccharose.

The course of the synthesis is shown in FIG. 12 on the basis of thin-layer chromatograms, where under a) the oligofructosylation of L-Fuc-Fru (tracks 1, 5, 9, 13), of L-Gal-Fru-Fru (tracks 2, 6, 10, 14), of L-Xyl-Fru (tracks 3, 7, 11, 15), and of L-Glu-Fru (tracks 4, 8, 12, 16) are branched after 0 minutes (tracks 1 to 4), after 5 minutes (tracks 5 to 8), after 10 minutes (tracks 9 to 12), and after 20 minutes (tracks 13 to 16), and under b) the same reactions [ . . . ] after 30 minutes (tracks 1 to 4), after 60 minutes (tracks 5 to 8), after 120 minutes (tracks 9 to 12), and after 240 minutes (tracks 13 to 16), and under c) after 1220 minutes, in track 1 L-Fuc-Fru, track 2 L-Gal-Fru-Fru, track 3 L-Xyl-Fru, and track 4 L-Glu-Fru. These results were confirmed by ion exchange chromatography (Dionex).

### EXAMPLE 9

### Transfer of the Aldopyranoside Group from a Saccharose Analogue onto a Peptide or Natural Material

FIG. 13 schematically shows the transfer of an aldopyranoside group for the example of the glucosyl group onto a compound containing hydroxyl groups, which can be a peptide or some other natural material. For easier handling and monitoring of the transfer reaction, this acceptor molecule is coupled to a polymeric carrier. According to the present invention, the aldopyranoside group, shown here in the example as a glucosyl group, is to be replaced by a derivate of the glucosyl group or another aldopyranose.

The catalysis is enabled by a modified dextrane sucrase as glycosyl transferase, which transfers, once or multiple times, the aldopyranoside group onto the hydroxyl group of the peptide or natural material. The construction of the oligosaccharide on the peptide or natural material is controlled in that, following a limited reaction time, a first saccharose analogue that contains a first aldopyranoside group is washed in order to terminate the transfer reaction of the first aldopyranoside group after a prespecified time. In this way, the desired number of transferred first aldopyranoside groups can be predetermined on the basis of the reaction time. In a second reaction step, a second aldopyranoside group can be transferred at the time in which the glycosyl transferase is provided with a second saccharose analogue as a co-substrate having the second aldopyranoside group. After repeated washing in order to remove the second co-substrate, additional identical or different aldopyranoside groups can be built up in a specific manner by conversion with the respective saccharose analogue, which has a particular aldopyranoside group.

For the separation of the peptide or natural material provided with an oligosaccharide from the polymeric carrier, the binding molecule was hydrolyzed and photometrically detected.

For the analysis of the oligosaccharide chain that was synthesized on the peptide or natural material, NMR or MS can be used immediately, or, alternatively, the oligosaccharide can be analyzed after hydrolysis of the oligosaccharide by the peptide or natural material. The hydrolysis of the oligosaccharide by the peptide or natural material can take place using aqueous caustic soda [or: lye], acid or glycosidases. The oligosaccharide can be spectrophotometrically analyzed, for example through reaction with glucose isomerase and hexokinase, glucose-6-phosphate-dehydrogenase in the presence of NADP and ATP, so that NADPH₂ can be measured spectrophotometrically. Through this analysis, the substrate specificity of the transferase that is used can be determined, in particular given a mixture of saccharose analogues as a co-substrate.

### EXAMPLE 10

### Synthesis of Oligosaccharides by Transferring the Aldopyranoside Group from β-D-fructosyl-α-D-galactoside

This example shows schematically, in FIG. 14, the synthesis of an oligo- or poly-aldopyranosyl fructoside. Thus, β-D-fructosyl-α-D-galactoside, produced according to Ex. 3 from saccharose and galactose as a saccharose analogue, is converted with a glycosyl transferase that was produced according to Example 1 through mutagenesis and screening from a glycosyl transferase gene. The analysis yielded the result that the galactoside group was transferred with accompanying release of fructose, so that an oligogalactofructoside was obtained.

As FIG. 15 shows schematically, the catalysis by glycosyl transferase mutations that are to be used according to the present invention, which can be obtained for example according to Example 1, transfers in each case the aldopyranoside group, which is not a glucoside, of a saccharose analogue onto an oligosaccharide, with accompanying release of the fructosyl group. In this way, from the saccharose analogue, here Gal-Fru, with the dextrane sucrase from Bacillus subtilis an oligoaldopyranosyl fructoside, here an oligogalactosyl fructoside, can be produced.

### EXAMPLE 11

### Synthesis of Oligosaccharides Through Transfer of the Ketofuranosyl Group from β-D-furanosyl-alpha-D-glucoside

The saccharose analogue β-D-furanosyl-alpha-D-glucoside was obtained corresponding to Example 3 through the conversion of saccharose with a ketofuranose. Through catalysis of fructosyl transferase variants that are obtainable according to Example 1, with the release of glucose the ketofuranosyl group can be transferred onto an acceptor molecule, for example an oligosaccharide. A scheme of this transfer reaction is shown in general in FIG. 16. As a product, a glucosyl-oligo- or -polyfuranoside is obtained.

### EXAMPLE 12

### Chromatographic Separation of Saccharides

For the separation of educts and products, both in the enzymatic synthesis of saccharides analogues and after the synthesis of oligosaccharides, remaining educts and product were separated through chromatography using ion exchangers (commercially available Purolite, Lavatite, Amberlite XAD). The elution took place with distilled water (Berensmeier et al., Separation and Purification Technology, 38, 129-138 (2004)).

1. A method for the synthesis of oligosaccharides or for glycosylation through the enzyme-catalyzed transfer of the aldopyranoside group or of the furanosyl group from a β-D-ketofuranosyl-α-D-aldopyranoside onto an acceptor molecule, characterized in that the β-D-ketofuranosyl-α-D-aldopyranoside is a saccharose analogue in which the ketofuranosyl group is different from the fructosyl group, or the D-aldopyranoside group is different from the glucose group.

2. The method as recited in claim 1, characterized in that instead of a saccharose analogue a raffinose analogue is used.

3. The method as recited in claim 1, characterized in that the saccharose analogue or raffinose analogue is synthesized through enzymatic conversion of the ketofuranose or of the aldopyranose with saccharose or raffinose.

4. The method as recited in claim 1, characterized in that the pyranoside group is selected from the group comprising the ribose, arabinose, xylose, lyxose, allose, altrose, galactose, derivatized glucose, mannose, gulose, idose, talose, fucose, rhamnose, 2-N-acetylglucosamine, 2-N-acetylgalactosamine, 2-deoxyglucose, 3-deoxyglucose, 4-deoxyglucose, 6-deoxyglucose, 2-deoxygalactose, 3-deoxygalactose, 3-ketoglucose, 4-ketoglucose, sialinic acid, N-acetyl-neuraminic acid, maltose, isomaltose, melibiose, cellobiose, lactose, or tagatose group, and other pentoses, hexoses, or heptoses that are to be glycosidically bound, each, independently, in the D or L configuration, L-glucose, and substituted derivates of the above-named groups, as well as mixtures thereof.

5. The method as recited in claim 1, characterized in that the β-D-ketofuranosyl group is selected from the group that comprises the ribulose, xylulose, derivatized fructose, ribulose, psicose, sorbose, and tagatose groups, and other pentoses, hexoses, heptoses, and substituted derivates of the above-named groups, that are to be glycosidically bound to the C1 of the glycoside group, as well as mixtures thereof.

6. The method as recited in claim 1, characterized in that the enzyme-catalyzed transfer is effected by a glycosyl transferase that is specific for the aldopyranoside.

7. The method as recited in claim 6, characterized in that the glycosyl transferase is a glucane sucrase, β-glucosidase, or a mutation thereof.

8. The method as recited in claim 1, characterized in that the enzyme-catalyzed transfer is effected by a glycosyl transferase that is specific for the ketofuranosyl group.

9. The method as recited in claim 8, characterized in that the glycosyl transferase is a fructosyl transferase or a mutation thereof.

10. The method as recited in claim 1, characterized in that the acceptor molecule has hydroxyl groups or thiol groups.

11. The method as recited in claim 10, characterized in that the acceptor molecule is selected from the group that comprises the carbohydrates, saccharides, steroids, terpenes, polyketides, hydroxy amino acids, hydroxy nitriles, metabolic products of microorganisms, diglycerides, ceramides, phenols, flavonoids, peptides containing asparagine, purines, pyrimidines, benzimidazols, nicotinic acid amide, and compounds containing these.

12. The method as recited in one of claims 10, characterized in that the acceptor molecule is the saccharose analogue.

13. The method as recited in one of claims 10, characterized in that the acceptor molecule is a β-D-ketofuranosyl-α-D-aldopyranoside or a β-D-ketofuranosyl-β-L-aldopyranoside.

14. The method as recited in claim 1, characterized in that the transferase is immobilized.

15. A method for synthesizing an oligosaccharide, characterized by multiple step-by-step application of a method as recited in claim 1, in which, in step-by-step fashion, each of various saccharose analogues or a mixture thereof are used.

16. A pyranosyloligofructoside or pyranosylpolyfructoside, obtainable through enzymatically catalyzed transfer of fructosyl groups from fructosyl aldopyranoside, the pyranoside group being selected from the group comprising ribose, arabinose, xylose, lyxose, allose, altrose, galactose, derivatized glucose, mannose, gulose, idose, talose, fucose, rhamnose, 2-N-acetylglucosamine, 2-N-acetylgalactosamine, 2-deoxyglucose, 3-deoxyglucose, 4-deoxyglucose, 6-deoxyglucose, 2-deoxygalactose, 3-deoxygalactose, 3-ketoglucose, 4-ketoglucose, sialinic acid, N-acetyl-neuraminic acid, maltose, isomaltose, melibiose, cellobiose, lactose, or tagatose groups, and other tetroses, pentoses, hexoses, or heptoses that are each to be glycosidically bound independently in the D or L configuration, and substituted derivates of the above-named groups, and being obtainable through enzymatic conversion of the aldopyranose with saccharose.

17. The pyranosyloligofructoside or pyranosylpolyfructoside as recited in claim 16, characterized in that the enzymatically catalyzed transfer and/or the enzymatic conversion of the aldopyranose with saccharose is catalyzed by fructosyl transferase.

18. The pyranosyloligofructoside or. pyranosylpolyfructoside as recited in claim 16, characterized by a number of fructosyl groups in the range from 2 to 10⁶, 2 to 100, preferably 5 to 20.


SEQUENCE LISTING (5 sequences)

SEQ ID NO: 1 - protein, 15 aa, Streptococcus oralis
YIFVRAHDDEVQTVI

SEQ ID NO: 2 - protein, 15 aa, Streptococcus oralis
YIFVRAHDREVQTVI

SEQ ID NO: 3 - protein, 15 aa, Streptococcus oralis
YIFVRAHDSEIQTVI

SEQ ID NO: 4 - protein, 15 aa, Streptococcus oralis
YIFVRAHDSEVQTVI

SEQ ID NO: 5 - protein, 1575 aa, Streptococcus oralis
MMEKKIHYKMHKVKKNWVAIALTTLALIVAPKVLGLESGVVHADDVKQVVVQEPATAQTSGPGQQTPAQAKIASEQEAEKVTPADKVTDDVAASEKPAKPAENTEATVQTNAQEPAKPADTKEASTEKAAVAEEVKAANAITEIPKTEVADQNKQARPTTAQDQEGDKREKTAVEDKIVANPKVAKKDRLPEPGSKQGAIAERMVADQAQPAPVNADHDDDVLSHIKTIDGKNYYVQDDGTVKKNFAVELNGRILYFDAETGALVDSNEYQFQQGTSSLNNEFSQKNAFYGTTDKDIETVDGYLTADSWYRPKFILKDGKTWTASTETDLRPLLMAWWPDKRTQINYLNYMNQQGLGAGAFENKVEQALLTGASQQVQRKIEEKIGKEGDTKWLRTLMGAFVKTQPNWNIKTESETTGTKKDHLQGGALLYTNNEKSPHADSKFRLLNRTPTSQTGTPKYFIDKSNGGYEFLLANDFDNSNPAVQAEQLNWLHYMMNFGSIVANDPTANFDGVRVDAVDNVNADLLQIASDYFKSRYKVGESEEEAIKHLSILEAWSDNDPDYNKDTKGAQLAIDNKLRLSLLYSFMRNLSIRSGVEPTITNSLNDRSSEKKNGERMANYIFVRAHDSEVQTVIADIIRENINPNTDGLTFTMDELKQAFKIYNEDMRKADKKYTQFNIPTAHALMLSNKDSITRVYYGDLYTDDGQYMEKKSPYHDAIDALLRARIKYVAGGQDMKVTYMGVPREADKWSYNGILTSVRYGTGANEATDEGTAETRTQGMAVIASNNPNLKLNEWDKLQVNMGAAHKNQYYRPVLLTTKDGISRYLTDEEVPQSLWKKTDANGILTFDMNDIAGYSNVQVSGYLAVWVPVGAKADQDARTTASKKKNASGQVYESSAALDSQLIYEGFSNFQDFATRDDQYTNKVIAKNVNLFKEWGVTSFELPPQYVSSQDGTFLDSIIQNGYAFEDRYDMAMSKNNKYGSLKDLLNALRALHSVNIQAIADWVPDQIYNLPGKEVVTATRVNNYGTYREGAEIKEKLYVANSKTNETDFQGKYGGAFLDELKAKYPEIFERVQISNGQKMTTDEKITKWSAKYFNGTNILGRGAYYVLKDWASNDYLTNRNGEIVLPKQLVNKNSYTGFVSDANGTKFYSTSGYQAKNSFIQDENGNWYYFDKRGYLVTGAHEIDGKHVYFLKNGIQLRDSIREDENGNQYYYDQTGAQVLNRYYTTDGQNWRYFDAKGVMARGLVKIGDGQQFFDENGYQVKGKIVSAKDGKLRYFDKDSGNAVINRFAQGDNPSDWYYFGVEFAKLTGLQKIGQQTLYFDQDGKQVKGKIVTLSDKSIRYFDANSGEMAVGKFAEGAKNEWYYFDKTGKAVTGLQKIGKQTLYFDQDGKQVKGKVVTLADKSIRYFDADSGEMAVGKFAEGAKNEWYYFDQTGKAVTGLQKIDKQTLYFDQDGKQVKGKIVTLSDKSIRYFDANSGEMATNKFVEGSQNEWYYFDQAGKAVTGLQQVGQQTLYFTQDGKQVKGKVVDVNGVSRYFDANSGDMARSKWIQLEDGSWMYFDRDGRGQNFGRN